(19) United States
(12) Patent Application Publication
(10) Pub. No.: US 2003/0027772 A1
(43) Pub. Date: Feb. 6, 2003

(54) Composition containing at least one inhibitor of the enzyme 3-beta-HSD

(75) Inventor: Lionel Breton, Versailles (FR)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(73) Assignee: L'OREAL, Paris (FR)

(21) Appl. No.: 10/157,152

(22) Filed: May 30, 2002

(30) Foreign Application Priority Data
May 30, 2001 (FR) 01 07102

(51) Int. Cl. A61K 31/7048; A61K 31/57; A61K 31/56; A61K 31/473; A61K 31/353

(52) U.S. Cl. 514/27; 514/179; 514/456; 514/182; 514/284

(57) ABSTRACT

The invention relates to a composition for topical application or oral administration, containing an effective quantity of at least one inhibitor of the enzyme 3β-hydroxysteroid dehydrogenase. This composition is designed in particular for the treatment of certain effects of chronological ageing and photo-ageing of the skin.

### FIELD OF THE INVENTION

The invention relates to a composition, preferably a cosmetic and/or dermatological and/or pharmaceutical composition for topical application or oral administration, containing an efficacious quantity of at least one inhibitor of the enzyme 3,β-hydroxysteroid dehydrogenase (3βHSD). This composition is designed for the treatment of certain signs of cutaneous ageing, endogenous and/or exogenous (chronological and/or photo-ageing).

### BACKGROUND OF THE INVENTION

Cutaneous ageing results from the effects of intrinsic and extrinsic factors on the skin. Clinically, the signs of ageing result in the appearance of wrinkles or lines, in a slackening of the cutaneous and subcutaneous tissues, in a loss of cutaneous elasticity, in an atony of the texture of the skin, and in the yellowing of the skin which becomes dull and lacks lustre.

Often spots of pigmentation, telangiectases and an elastosis are observed on the areas of the skin which have been exposed to the sun throughout life, essentially the face, the throat, neck and shoulders, the hands and the forearms.

Some of these signs are more particularly linked to intrinsic or physiological ageing, i.e. ageing linked to age. Intrinsic ageing is also called chronological ageing.

Other signs are more specific for extrinsic ageing, i.e. ageing caused in a general manner by the environment; it relates more particularly to photo-ageing due to exposure to the sun, light or to any other radiation.

The changes in the skin resulting from chronological ageing are the consequence of genetically programmed senescence in which endogenous factors are implicated. This intrinsic ageing causes in particular a retardation of the renewal of the cells of the skin. Histologically, the skin generally becomes thinner, at both the epidermic and dermic levels. In addition, a slackening of the cutaneous and subcutaneous tissues is observed which results in an atonic texture of the skin, a slackening of the cutaneous microrelief, reduced cutaneous firmness and an overall flabby skin. The density of the fibrous macromolecules of the dermis (elastin and collagen) is diminished.

A reduction of the endogenous level of DHEA can also be observed among some people.

Extrinsic ageing leads, in its case, to histopathological changes such as the excessive accumulation of non functional elastic material in the upper dermis and the degeneration of the collagen fibres. These histopathological changes also lead to an impairment of the dermis and to a reduction of the firmness and elasticity of the skin.

In both the chronological and extrinsic cases of ageing it is the collagen fibres in particular which are impaired. These collagen fibres ensure the elasticity of the dermis. The collagen fibres are constituted of fibrils cemented to each other, thus forming more than 10 types of different structures. The strength of the dermis is due in large measure to the interlacing of the collagen fibres packed against each other in all directions. The collagen fibres participate in the elasticity and tonicity of the skin and/or mucous membranes.

The collagen fibres are continually renewed but this renewal slows down with age and this leads to a thinning of the dermis. This thinning of the dermis is also due to pathological causes like for example the hypersecretion of corticoid hormones, certain diseases or even certain vitamin deficiencies (the case of vitamin C in scurvy). It is also acknowledged that extrinsic factors like ultraviolet radiation, tobacco or certain treatments (glucocorticoids, vitamin D and derivatives for example) also have an effect on the skin and on its collagen level.

The invention relates particularly to the correction of the signs of chronological ageing. This invention focuses on the activation of cellular renewal and/or on an improvement in the density of the fibrous macromolecules of the dermis. A further object of the invention is the maintenance of the engogenous level of DHEA at the level of the dermis and epidermis, among people for which this DHEA level tends to diminish.

The invention also relates to the correction of the wrinkles and lines of the skin, to the correction of the slackening of the cutaneous and subcutaneous tissues and to an improvement in the lustre of the skin.

At present, there exist many compositions which claim to treat wrinkles and lines of the skin or strengthen the cutaneous tissues, but these compositions only treat these morphological disorders incompletely and transiently.

Compositions have certainly already been proposed for treating more efficaciously and in the long term wrinkles and lines, strengthening the cutaneous tissues and giving lustre to an atonic skin, and the exogenous supply of steroid hormones, and in particular the supply of dehydroepi-androsterone (DHEA) and/or its derivatives (like for example dehydroepi-androsterone sulfate or S-DHEA) in cosmetic and/or pharmaceutical and/or dermatological compositions lead to satisfactory results in the treatment of these adverse effects to the cutaneous tissue.

By exogenous supply of steroid hormones is meant in the context of the application any supply of steroid hormones derived from the exterior of the organism but which might be secreted by the organism.

There exist many patents describing cosmetic or dermatological compositions for topical application containing dehydroepiandrosterone and/or its derivatives. For example, the American patent U.S. Pat. No. 5,989,568 describes the use of dehydroepi-androsterone sulfate in a topical composition for the treatment of wrinkles, lines and/or for combating cutaneous and/or subcutaneous slackening and/or brightening the lustre of the skin.

However, the major disadvantage of these compositions consists precisely in the fact that they contain steroid hormones which are subject to specific regulatory restraints with regard to their use.

### OBJECT OF THE INVENTION

Thus to-day there exists in the state of the art a great need for a method making it possible to treat efficaciously and in the long term signs of cutaneous ageing not involving the exogenous supply of steroid hormones and, in particular, of DHEA. Hitherto, no one has imagined the positive regulation of the tissue level of RHEA without an external supply of this molecule.

The present invention aims precisely to satisfy such a need, and does so as explained below.

### SUMMARY OF THE INVENTION

Now, the applicant has made the surprising discovery that the enzyme 3, β-hydroxysterold dehydrogenase (also called 3β-HSD) is expressed at the level of the epidermis and dermis in human beings, i.e. at the level of the human epidermal keratinocytes and dermal fibroblasts.

By expression of the enzyme 3β-HSD is meant in the context of the application the expression of the mRNA of 3β-HSD which after translation leads to the synthesis of the protein: the enzyme.

It is known that the enzyme 3β-HSD is implicated in the catabolism of the endogenous DHEA to androstedione.

A diminution of the activity and/or expression of this enzyme is consequently equivalent to retarding the degradation of DHEA and to maintaining an adequate endogenous level of DHEA at the level of the dermis and epidermis without exogenous supply of DHEA.

The present invention thus provides a composition, preferably a cosmetic and/or dermatological and/or pharmaceutical composition for topical application or oral administration, characterized in that it contains an efficient or effective quantity of at least one inhibitor of the enzyme 3β-hydroxysteroid dehydrogenase. Methods for making such compositions, and their use, also make up part of the invention.

The topical application of such a composition makes it possible to exert a positive regulation on the endogenous cutaneous level of DHEA and/or its derivatives (such as in particular DHEA sulfate) and maintain it at a physiological level or higher level.

The composition preferably contains a cosmetically or dermatologically acceptable medium, i.e. compatible with the cutaneous tissues. Thus, the composition can be applied to any human body.

### BRIEF DESCRIPTION OF THE DRAWING

FIG. 1 presents the results of an agarose gel electrophoresis after reverse transcription on RNA of different cell samples derived from normal skins or skins reconstructed in vitro.

### DETAILED DESCRIPTION OF THE INVENTION

The 3β-HSD inhibitor according to the invention is preferably present in the composition in a proportion of 0.0000001 to 25% by weight, and more preferably from 0.00001 to 10% by weight with respect to the total weight of the composition.

Advantageously, the inhibitor of 3p-HSD may be selected from the isoflavones and its derivatives, and preferably from daidzein and genistein, as well as their isoflavan derivatives: equol or 4′,7-dihydroxy-isoflavan and 5-hydroxyequol or 4′,5,7-trihydroxyisoflavan, respectively.

A preferred isoflavone according to the invention is genistein and/or its flavan equivalent: 5-hydroxyequol or 4′,5,7 trihydroxy-isoflavan.

Also useful as inhibitors of 3β-HSD according to the invention are the compounds selected from Epostane (WIN 32729), Trilostane (WIN 24540), 11-α-bromoacetoxy-progesterone, 11-alkyl derivatives of progesterone, 3 or 4 ketosteroid derivatives, RU486 and N,N-dimethyl-4-methyl-4-3-oxo-4-aza-5-androstane- 17-β-carboxamide (4-MA); cholesterol, its esters and derivatives, 16-α-hydroxydehydroepiandrosterone, 17-β-estradiol, ethynylestradiol, chloramidone acetate, cyanotrimethylandrostenolone.

Naturally, it is possible to combine the inhibitor(s) of 3β-HSD according to the invention with natural or synthetic hormones selected from the estrogenic, progestative and androgenic hormones like progesterone, testosterone, broparestrol, estrone, pregnenolone acetate, pregnenolone, 17′-β-hydroxyprogesterone, testosterone propionate, androstenedione and androstanediols.

It is also possible to combine the inhibitor of 3β-HSD according to the invention with molecules and/or mimetic plant extracts of estradiol and/or progesterone.

By mimetic plant extract is meant, in the context of the application, any plant extract capable of mimicking the action of estradiol and/or progesterone for the correction of the effects of skin ageing, without the disadvantages associated with the use of the hormones.

Finally, it is also possible to combine the inhibitor(s) of 3β-HSD, and more particularly the isoflavone(s) according to the invention, with dehydroepiandrosterone (DHEA) and/or derivatives of DHEA, and in particular DHEA sulfate, known to possess anti-ageing properties, and more particularly possessing marked activity in the fields of the treatment of wrinkles and/or lines, combating cutaneous or subcutaneous slackening and/or brightening of lustre.

According to the invention it is also possible to combine the inhibitor(s) of 3β-HSD with DHEA derivatives such as 7 OH-DHEA, 7 keto-DHEA and 3β-acetoxy-7-oxo-DHEA.

The compositions usable in the invention may be available in all galenic forms conventionally used for a topical application and in particular in the form of an aqueous, alcoholic or aqueous-alcoholic solution or suspension or an oily suspension or a solution or dispersion of the lotion or serum type, an emulsion of liquid or semi-liquid consistency of the milk type, obtained by dispersion of a fatty phase in an aqueous phase (O/W) or the reverse (W/O), or a suspension or emulsion of soft consistency of the cream type (O/W) or (W/O), or an aqueous or anhydrous gel, an ointment, a free-flowing or compacted powder to be used as such or to be incorporated in an excipient, or in the form of microcapsules or microparticles, or vesicular dispersions of the ionic and/or non-ionic type.

The compositions used in the process according to the invention may contain at least one liquid or solid fatty material.

As fatty materials according to the invention, mention may be made of the mineral oils (vaseline oil), plant oils (liquid fraction of karite butter, sunflower oil), animal oils (perhydrosqualene), synthetic oils (Purcellin oil), siliconized oils (cyclomethicone) and fluorinated oils (perfluoropolyethers). Fatty alcohols, acids (stearic acid), waxes (paraffin, carnauba, beeswax) may also be used. a fatty materials.

The oils, the emulsifiers and coemulsifiers used in the composition in the form of an emulsion are selected from those conventionally used in the cosmetic or dermatological field.

As emulsifiers usable in the invention, mention may be made for example of glycerol stearate, polysorbate 60 and the mixture of PEG-6/PEG632/glycol stearate sold under the name Tefose® 63 by the Gattefosse company.

The emulsifier and coemulsifier are present in the composition in a proportion ranging from 0.3% to 30% by weight, and preferably from 0.5 to 20% by weight with respect to the total weight of the composition.

In a known manner, the composition according to the invention may also contain adjuvants usually used in the cosmetic, pharmaceutical or dermatological field, such as the hydrophilic or lipophilic gelling agents, the hydrophilic or lipophilic active agents, the preservatives, the antioxidants, the solvents, the perfume, the fillers, the filters, the bactericidal agents, the odour absorbents and the colouring matters.

The quantities of these different adjuvants are those conventionally used in the field under consideration, far example from 0.001 to 20% of the total weight of the composition.

Depending on their nature, these adjuvants may be introduced in the fatty phase, the aqueous phase and/or in tiny liquid spheres.

As solvents usable according to the invention, mention may be made of the lower alcohols, in particular ethanol and isopropanol, propylene glycol.

As hydrophilic gelling agents usable according to the invention, mention may be made of the carboxyvinyl polymers (carbomer), the acrylic copolymers such as the acrylate/alkylacrylate copolymers, the polyacrylamides, the polysaccharides such as hydroxypropylcellulose, the natural gums and the clays.

As lipophilic gelling agents usable according to the invention, mention may be made of the modified clays such as bentones, the metal salts of fatty acids such as the aluminium stearates and hydrophobic silica, or also ethylcellulose and polyethylene.

As lipophilic or hydrophilic active agents usable in the invention for the purpose of improving the treatment of wrinkles and lines, the control of cutaneous and/or subcutaneous slackening and/or the lustre of the skin, mention may be made for example of the retinoids (retinol and its esters, retinal, retinoic acid and its derivatives, retinoids, in particular two described in the documents FR-A-2570377, EP-A-199636, EP-325540, EPA-402072), the α-hydroxyacids (glycolic, lactic, malic, citric, tartaric, mandelic)), the β-hydroxyacids (salicylic acid and its derivatives, particularly alkylated), the α-ketoacids, the β-ketoacids, the peroxides such as benzoyl peroxide, the vitamins especially the vitamins E, F, the anti-radical active agents such as superoxide dismutase, selenium, zinc, the carotenoids, in particular betacarotene, lycopene, lutein, zeaxanthin.

The invention also relates to a process for correcting the effects of chronological ageing and/or photo-ageing of the skin, which consists of applying the composition according to the invention to the skin.

More particularly, the invention relates to a process for activating the epidermal and/or dermal cellular renewal and/or improving the density of the fibrous macromolecules of the dermis, and/or regulating the synthesis and/or degradation of the matrix proteins of the dermis, and/or correcting the slackening of the cutaneous and subcutaneous tissues, and/or brightening the lustre of the skin and/or diminishing the wrinkles and/or lines, which consists of applying the composition according to the invention to the skin.

The object of the invention is also a method for regulating the pigmentation processes of the skin and exoskeleton, and/or the seborrhea and/or the growth of hair and/or body hair and/or the nails, which consists of applying the composition according to the invention to the skin and/or the exoskeleton.

Finally, the invention also relates to the use in a cosmetic and/or dermatological and/or pharmaceutical composition for topical application of an effective amount of at least one inhibitor of the enzyme 3β-hydroxysteroid dehydrogenase as corrective agent of the effects of chronological ageing and/or photo-ageing of the skin, and more particularly as:

activation agent of cell renewal and/or agent for the improvement of the density of the fibrous macromolecules of the dermis, and/or

regulatory agent of the synthesis and/or degradation of the matrix proteins of the dermis, and/or

corrective agent of the slackening of the cutaneous and subcutaneous tissues, and/or

brightening of the lustre of the skin and/or reduction of the wrinkles and lines of the skin, and/or

regulatory agent of the pigmentation processes of the skin and exoskeleton and/or seborrhea and/or the growth of hair and/or body hair and/or nails,

the effective amount being that amount sufficient to accomplish one or more of these effects.

The following samples illustrate the invention. In these examples, the proportions indicated are percentages by weight.

### EXAMPLES

### Example 1

Demonstration of the Expression of the mRNA of 3β-HSD in the Epidermis and Dermis

It is desirable to verify the expression of the enzyme 3β-hydroxysteroid dehydrogenase (3β-HSD) in different cellular models derived from human skin: cell culture of keratinocytes, cell culture of fibroblasts, reconstructed skins or surgical samples of human skin.

To this end, the expression of the mRNA of the 3β-HSD will be investigated by the RT-PCR method. RT-PCR is a method which enables the presence of the mRNA coding for the desired protein (in this case, 3β-HSD) to be detected, even in low amounts.

Procedure

1. Preparations of total RNAs from 4 different origins (keratinocyte cell culture, fibroblast cell culture, reconstructed skins or surgical samples of human skin).

Total RNA of normal human dermal fibroblasts (NHDF) cultured as a monolayer in MEM/M199 medium supplemented with 1% fetal calf serum.

Total RNA of normal human epidermal keratinocytes (NHEK) cultured as monolayers in SFM medium (Gibco) without calcium supplementation (medium poor in calcium, 0.09 mM Ca⁺²).

Total RNA of reconstructed epidermis SkinEthic (SkinEthic Laboratories, Nice, France), 17 days old, cultured in SkinEthic medium.

4 Total RNA of normal human cutaneous epidermis (mammary plasty).

The frozen cells and tissues were lysed and the total RNAs were extracted with the aid of TriReagent (Sigma) according to the protocol recommended by the supplier.

2. Analysis

The total RNAs were purified (extraction of the total RNAs) starting from the 4 experimental conditions (keratinocyte cell culture, fibroblast cell culture, reconstructed skins or surgical samples of human skin).

Starting from the total RNA, the complementary DNA (cDNA) of the different mRNAs (present in the total purified RNA) is synthesized by reverse transcription with an oligo primer (dT).

A specific sequence (302 base pairs) corresponding to the sequence of 3β-HSD is amplified by PCR (Polymerise Chain Reaction) using specific oligonucleotides.

The amplified fragments are then analysed by electrophoresis on agarose gel (1.7%) in the presence of ethidium bromide. The photographs were obtained on GelPrint 2000i (BioPhotonics Corp.).

3. Results

A unique fragment corresponding to the size of the expected 302 base pairs fragment (which corresponds to 3β-HSD) was clearly detected in all of the samples tested (cultures, reconstructed skins or surgical samples).

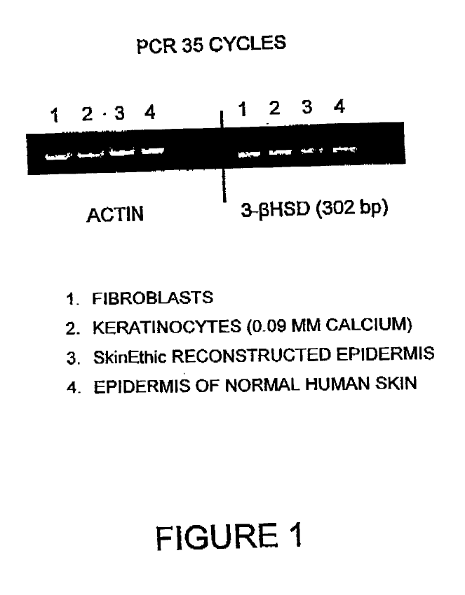

The results are illustrated in FIG. 1.

The mRNA of 3β-HSD is thus expressed ire both the epidermis (keratinocytes) and in the dermis (fibroblasts).

This observation makes it possible to conclude that the protein 3β-HSD is probably present in the skin.

### Example 2

Composition 1:
Soft capsules/Administration by the oral route

| | |
|---|---|
| Excipients: | |
| Soya oil | 40 mg |
| Wheat germ oil | 85 mg |
| Soya lecithins | 25 mg |
| Vitamins/antioxidants: | |
| Natural tocopherols | 3 mg |
| Vitamin C | 50 mg |
| Lycopene | 5 mg |
| Constituents: | |
| Genistein | 25 mg |

Composition 2:
Care cream (oil in water emulsion) to activate the epidermal cell
renewal and/or improve the density of the fibrous macromolecules.

| Genistein | | 0.1% |
|---|---|---|
| 7OH-DHEA | | 0.2% |
| Glycerol stearate | | 2.00% |
| Polysorbate 60 (Tween 60 ® sold by the ICI company) | | 1.00% |
| Stearic acid | | 1.40% |
| Triethanolamine | | 0.70% |
| Carbomer | | 0.40% |
| Liquid fraction of karite butter | | 12.00% |
| Perhydrosqualene | | 12.00% |
| Antioxidant | | 0.05% |
| Perfume | | 0.50% |
| Preservative | | 0.30% |
| Water | qsp | 100.00% |

Composition 3:
Gel for the skin designed to regulate the lustre
of the complexion and the wrinkles

| Daidzein | | 0.5% |
|---|---|---|
| Hydroxypropylcellulose | | 1.00% |
| (Klucel H ® sold by the Hercules company) | | |
| Antioxidant | | 0.05% |
| Isopropanol | | 40.00% |
| Preservative | | 0.30% |
| Water | qsp | 100.00% |

Composition 4:
Gel to correct the slackening of the cutaneous
and subcutaneous tissues, In particular by regulating
the synthesis and/or degradation of the matrix proteins

| Genistein | | 0.8% |
|---|---|---|
| Hydroxypropylcellulose | | 1.00% |
| (Klucel H ® sold by the Hercules company) | | |
| Antioxidant | | 0.05% |
| Lidocaine hydrochloride | | 2.00% |
| Isopropanol | | 40.00% |
| Preservative | | 0.30% |
| Water | qsp | 100.00% |

Composition 5:
Care cream (oil/water emulsion) to regulate the seborrhea

| 5 Hydroxyequol (4′,5,7-trihydroxy-isoflavan) | | 0.2% |
|---|---|---|
| Glycerol stearate | | 2.00% |
| Polysorbate 60 (Tween 60 ® sold by the ICI company) | | 1.00% |
| Stearic acid | | 1.40% |
| n-Actinal-5-salicylic acid | | 0.50% |
| Triethanolamine | | 0.70% |
| Carbomer | | 0.40% |
| Liquid fraction of karite butter | | 42.00% |
| Perhydrosqualene | | 12.00% |
| Antioxidant | | 0.05% |
| Perfume | | 0.50% |
| Preservative | | 0.30% |
| Water | qsp | 100.00% |

French patent application 01 07102 filed May 30, 2001, is incorporated herein by reference, as are all references, applications, patents, publications, articles, standards and texts referred to herein.

1. A composition suitable for topical application or oral administration, comprising an effective amount of at least one inhibitor of the enzyme 3β-hydroxysteroid dehydrogenase to treat or prevent at least one sign of cutaneous ageing.

2. The composition according to claim 1, wherein the inhibitor of the enzyme 3β-hydroxysteroid dehydrogenase is present in an amount of 0.00001 to 10 % by weight with respect to the total weight of the composition.

3. The composition according to claim 1, wherein the inhibitor of the enzyme 3β-hydroxysteroid dehydrogenase is selected from the isoflavones and isoflavans.

4. The composition according to claim 1, comprising daidzein or genistein.

5. The composition according to claim 1, comprising Equol or 5-hydroxy Equol.

6. The composition according to claim 1, wherein in that the inhibitor of the enzyme 3β-hydroxysteroid dehydrogenase is selected from the group consisting of Epostane, Trilostane, 11-α-bromoacetoxy-progesterone, 11-alkyl derivatives of progesterone, derivatives of 3 or 4 ketosteroids, RU486 and N,N-dimethyl-4-methyl-3-oxo-4-aza-5-androstane-17-β-carboxamide (4-MA).

7. The composition according to claim 1, wherein the inhibitor of the enzyme 3β-hydroxysteroid dehydrogenase is selected from the group consisting of cholesterol, its esters and its derivatives, 16-α-hydroxydehydroepiandrosterone, 17β-estradiol, ethynylestradiol, chloramidone acetate, and cyanotrimethylandrostenolone.

8. The composition according to claim 1, further comprising one or more natural or synthetic hormones selected from the group consisting of estrogenic, progestative and androgenic hormones.

9. The composition according to claim 1, further comprising a hormone selected from the group consisting of progesterone, estradiol, testosterone, broparestrol, estrone, pregnenolone acetate, pregnenolone, 17β-hydroxyprogesterone, testosterone propionate, androstene and the androstanediols.

10. The composition according to claim 1, further comprising molecules and/or mimetic plant extracts of estradiol and/or progesterone.

11. The composition according to claim 1, further comprising RHEA and/or RHEA sulfate and/or 7 OH-RHEA and/or 7-keto-DHEA and/or 3β-acetoxy-7-oxo-RHEA.

12. The composition according to claim 1, wherein said composition is in the form of an aqueous, alcoholic or aqueous-alcoholic solution or suspension or an oily suspension or a solution or dispersion of the lotion or serum type, an emulsion of liquid or semi-liquid consistency of the milk type, obtained by dispersion of a fatty phase in an aqueous phase (O/W) or the reverse (W/O), or a suspension or emulsion of soft consistency of ,the cream type (O/W) or (W/O), or an aqueous or anhydrous gel, an ointment, a free-flowing or compacted powder or in the form of microcapsules or microparticles, or vesicular dispersions of the ionic and/or nonionic type.

13. The composition according to claim 12, wherein the composition further comprises at least one liquid or solid fatty material selected from the group consisting of mineral oils, vegetable oils, animal oils, synthetic oils, siliconized oils or waxes, fluorinated oils, beeswax, Carnauba wax or paraffin wax, fatty acids, the fatty alcohols and the waxes.

14. The composition according to claim 1, further comprising one or more adjuvants selected from the group consisting of hydrophilic or lipophilic gelling agents, hydrophilic or lipophilic active agents, preservatives, antioxidants, solvents, perfumes, fillers, filters, bactericidal agents, odour absorbents and colouring matters.

15. The composition according to claim 1, further comprising a hydrophilic gelling agents selected from the group consisting of carboxyvinyl polymers, acrylic copolymers, polyacrylamides, polysaccharides, natural gums and clays.

16. The composition according to claim 1, further comprising an active agent selected from the group consisting of retinoids, α-hydroxyacids, β-hydroxy-acids, α-keto-acids, β-ketoacids, peroxides, vitamins, anti-radical active agents, selenium, zinc and β-carotene.

17. The composition according to claim 1, further comprising an agent selected from the group consisting of modified clays, metal salts of fatty acids, ethylcellulose and polyethylene.

18. A method of achieving cellular renewal and/or improving the density of the fibrous macromolecules of the dermis, comprising topically applying or orally administering to a human the composition of claim 1.

19. A method of regulating the synthesis and/or degradation of the matrix proteins of the dermis, comprising topically applying or orally administering to a human the composition of claim 1.

20. A method of treating the slackening of the cutaneous and subcutaneous tissues, comprising topically applying or orally administering the composition of claim 1 to a human.

21. A method for brightening the lustre of the skin and/or reducing wrinkles and lines of the skin, comprising topically applying or orally administering the composition of claim 1 to a human.

22. A method for regulating the pigmentation processes of the skin and exoskeleton and/or the seborrhea and/or the growth of hair and/or body hair and/or nails, comprising topically applying or orally administering the composition of claim 1 to a human.
